(19) United States
(12) Patent Application Publication
(10) Pub. No.: US 2025/0026708 A1
(43) Pub. Date: Jan. 23, 2025

(54) PROCESS FOR PREPARATION OF TRIHEPTANOIN

(71) Applicant: Navinta, LLC, Ewing, NJ (US)

(72) Inventors: Pankaj Vasudev Parmar, Vadodara (IN); John Muthiah Raja Jeyakumar, Vadodara (IN); Pratish Valjibhai Dadhaniya, Vadodara (IN); Miteshgir Kanchangir Goswami, Vadodara (IN)

(21) Appl. No.: 18/770,227

(22) Filed: Jul. 11, 2024

Related U.S. Application Data
(60) Provisional application No. 63/526,315, filed on Jul. 12, 2023.

(51) Int. Cl. C07C 67/08; C07C 51/295; C07C 51/44; C07C 67/56

(52) U.S. Cl. CPC C07C 67/08; C07C 51/295; C07C 51/44; C07C 67/56

(57) ABSTRACT

A process is provided for the preparation of triheptanoin having a purity of greater than about 99% as measured by HPLC and/or GC. The inventive process disclosed is simple, cost effective and provides preparation of highly pure triheptanoin without the use column chromatography. The inventive process for the preparation of stable and highly pure triheptanoin involves use of adsorbing agent and at least one organic solvent during purification.

### FIELD OF THE INVENTION

The present invention relates to a process for the preparation of triheptanoin. Particularly, the present invention relates to an economically feasible, facile, robust process for the preparation of pharmaceutically acceptable stable and highly pure triheptanoin without use of column chromatography. More particularly, the present invention relates to a process for the preparation of stable triheptanoin with purity of greater than about 99.5% and an acid value of greater than about 0.2 mg KOH/gm, which involves use of adsorbing agent and organic solvent during purification.

### BACKGROUND OF THE INVENTION

Triheptanoin as an oral liquid was approved by U.S. FDA in 2020 under the brand name of DOJOLVI®. It is a medium-chain triglyceride indicated as a source of calories and fatty acids for the treatment of pediatric and adult patients with molecularly confirmed long-chain fatty acid oxidation disorders (LC-FAOD).

The chemical name of triheptanoin is heptanoic acid, 1,1′,1″-(1,2,3 propanetriyl) ester. The empirical formula is C₂₄H₄₄O₆ and its molecular weight is 428.6 g/mol. The chemical structure of triheptanoin is shown below as Formula-I:

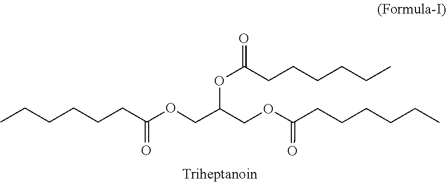 [SMILES: CCCCCCC(=O)OCC(COC(=O)CCCCCC)OC(=O)CCCCCC]

Triheptanoin was first disclosed in FR 1060166A, which describes the synthesis of fatty acid esters, in particular triheptanoate, from glycerol and heptanoic acid obtained from castor oil in the presence of sulfuric acid as a catalyst. There are drawbacks observed by following the process of this patent, e.g., it does not specify the purification method of crude triheptanoin, there is no disclosure about purity of the obtained triheptanoin, and there is formation of unavoidable impurities.

CA 620604 discloses the synthesis of triglycerides from a mixture of C6/C8/C10 fatty acids with an excess of 10% in acids relative to the weight of glycerin. The product is purified with a solution of sodium hydroxide (NaOH), and it is then washed, dried, bleached and filtered under a vacuum. There are drawbacks associated with the process of said patent, including the fact that the reaction is performed at high temperature (140-250° C.), which increases the cost and time, and no information about purity is provided.

FR 3112344 discloses the preparation of triheptanoin from the reaction between glycerol and heptanoic acid at higher temperature (210-230° C.) under high vacuum. The obtained crude triheptanoin is purified by using alumina without any solvent. The drawbacks associated with the process of this patent include that the obtained product is yellow and has a cloudy appearance, and the reaction is performed at higher temperature (210-230° C.), which increases the cost and time.

U.S. Pat. No. 9,447,016 discloses synthesis of triglycerides from reaction between fatty acids (C6-C12) and glycerol in presence of metal cation catalyst at higher temperature (i.e., 175° C.) under partial vacuum. This process has a number of drawbacks, including that the use of metal catalyst leads to the formation of unavoidable impurities and that the reaction is performed at higher temperature (175° C.), which increases the cost and time.

CN 113896631 discloses the preparation of triheptanoin from a reaction between glycerol and heptanoic acid by using N-chloro succinimide, tetramethyl thiourea and triethylamine in presence of acetonitrile as a solvent. This process has several drawbacks, including the use of excess reagents that leads to formation of byproducts and other impurities, and the use of column purification, which increases the cost.

Semak et al. (Eur. J. Lipid Sci. Technol. 2012, 114 (8), 889-895) reported the synthesis of triheptanoin from reaction between glycerol and heptanoic acid by using sulfonated charcoal catalyst and toluene as a solvent. The drawbacks of this process include that the preparation of sulfonated charcoal catalyst requires multiple operational steps, which increase the cost and time, and this process is not feasible at a commercial scale.

Ataide et al. (International Journal of Food Science and Technology, 2007, 42, 1504-1508) reported the synthesis of triheptanoin in the absence of both solvent and catalyst at high temperatures and under vacuum. The process involves two stages under high temperature and vacuum and a 50% molar excess of heptanoic acid. Triheptanoin was isolated in 79% yield after purification by column chromatography (silica gel, hexane/chloroform 1:1). There are a number of disadvantages associated with this method, including the use of purification by column chromatography, which results in yield loss, excess use of the starting material, i.e., heptanoic acid, and the fact that the synthesis was carried out under high partial vacuum (at 10 mmHg) and with a relatively long reaction time (for 29 h).

Hence, there is need to develop an alternative process for preparation of stable and highly pure triheptanoin, which is simple, commercially viable, cost effective, and provides stable and highly pure triheptanoin product.

The present invention provides a process for preparation of stable and highly pure triheptanoin, which fulfills aforesaid objectives.

### SUMMARY OF THE INVENTION

One embodiment of the present invention provides a process for preparation of stable and highly pure triheptanoin, which includes the steps of:
a) reacting glycerol with heptanoic acid using at least one organic solvent and at least one catalyst under reflux conditions;
b) isolating crude triheptanoin;
c) purifying crude triheptanoin using at least one adsorbing agent and optionally adding at least one second organic solvent; and
d) isolating stable and highly pure triheptanoin.

In certain embodiments of the present invention, step (c) further includes the steps of:
i) filtering followed by distilling the second organic solvent;
ii) optionally adding at least one third organic solvent and distilling the same;
iii) optionally adding a medium chain fatty acid;
iv) optionally removing traces of the organic solvents used in the step (a) and/or step (c); and
v) isolating stable and highly pure triheptanoin having an acid value of greater than about 0.2 mg KOH/gm.

Another embodiment of the present invention provides a process for preparation of stable and highly pure triheptanoin, which includes the steps of:
a) reacting glycerol with heptanoic acid using at least one first organic solvent and at least one acid catalyst under reflux conditions;
b) separating an organic layer from a reaction mass of step (a) and treating the reaction mass with at least one adsorbing agent and optionally adding at least one second organic solvent; and
c) isolating stable and highly pure triheptanoin.

In another embodiment of the present invention, a process for preparation of stable and highly pure triheptanoin is provided, including the steps of:
a) reacting glycerol with heptanoic acid using at least one first organic solvent and at least one acid catalyst under reflux conditions;
b) separate an organic layer from a reaction mass of step (a) and treating the reaction mass with at least one adsorbing agent and optionally adding at least one second organic solvent;
c) distilling the organic solvent(s) to obtain a residue;
d) stirring the obtained residue;
e) filtering the reaction mass; and
f) isolating stable and highly pure triheptanoin.

Yet another embodiment of the present invention provides a process for purification of triheptanoin, which includes treating crude triheptanoin compound with a suitable adsorbing agent and a suitable organic solvent.

A further embodiment of the present invention provides stable and highly pure triheptanoin by avoiding use of column chromatography.

Yet another embodiment of the present invention provides stable and highly pure triheptanoin by involving less formation of impurities.

In some embodiments, the stable and pure triheptanoin produced by the method has a purity of greater than about 99%, or greater than about 99.5%, or greater than about 99.7%, as measured by HPLC and/or GC.

In some embodiments, the stable and highly pure triheptanoin produced by the method comprises not more than about 0.05% of 1,2-diester impurity as measured by gas chromatography (GC) and/or high-performance liquid chromatography (HPLC).

In some embodiments, the method produces stable and highly pure triheptanoin, which comprises not more than about 0.05% of 1,3-diester impurity as measured by gas chromatography (GC) and/or high-performance liquid chromatography (HPLC).

In some embodiments, the method produces stable triheptanoin with high purity that has an acid value of the triheptanoin of greater than about 0.2 mg KOH/gm.

Yet another embodiment of the present invention provides a process for preparation of pure heptanoic acid from commercial grade heptanoic acid via formation of methyl heptanoate.

Yet another embodiment of the present invention provides a purification of heptanoic acid, which includes the steps of:
a) treating crude heptanoic acid with an aqueous solution of base followed by washing with at least one first organic solvent;
b) acidifying an aqueous layer of step (a) by using an acid followed by extraction in at least one second organic solvent and distilling the solvent to obtain crude heptanoic acid;
c) esterifying the crude heptanoic acid obtained in step (b) with an alcohol in presence of at least one catalyst;
d) distilling crude alkyl heptanoate as obtained in step (c) optionally under vacuum to obtain pure alkyl heptanoate having a content of any impurity below about 0.1%; and
e) hydrolyzing pure alkyl heptanoate as obtained in step (d) by using a base and at least one third organic solvent to obtain pure heptanoic acid having a purity of greater than about 99.5% and a content of any impurity below about 0.1%.

Other features and advantages of the present invention will become apparent from the following more detailed description, which illustrate, by way of example, the principle of the invention.

### DETAILED DESCRIPTION OF THE INVENTION

The phrase “water-immiscible organic solvent,” unless otherwise indicated, refers to an organic solvent which is insoluble in water in all proportions at standard temperature and pressure. Suitable water-immiscible organic solvents include, but are not limited to, toluene, xylene, benzene, cyclohexane, ethyl acetate, methyl tert-butyl ether, diisopropyl ether, heptane, hexane, isopropyl acetate, methyl acetate, 2-methyl tetrahydrofuran.

The term “catalyst” refers to a substance used to trigger a reaction and which is practically not consumed during this reaction. The catalyst typically used in esterification reaction can be an acid catalyst, a metal catalyst or an enzyme, or any catalyst well known to those skilled in the art.

The terms “comprising” and “comprises” mean the elements recited, or their equivalents in structure or function, plus any other element or elements which are not recited.

It is noted that, as used in the specification and the claims, the singular form “a,” “an,” and “the” comprises plural referents unless the context clearly indicates otherwise. For example, reference to a component in the singular is intended to comprise a plurality of components.

The terms “having” and “including” are to be construed as open ended. All ranges recited herein include the endpoints, including those that recite a range between two values. Whether so indicated or not, all values recited herein are approximate as defined by the circumstances, including the degree of expected experimental error, technique error, and instrument error for a given technique used to measure a value.

The term “about” is to be construed as modifying a term or value such that it is not an absolute. This term will be defined by the circumstances. This includes, at the very least, the degree of expected experimental error, technique error and instrument error for a given technique used to measure a value. In general, this term used in connection with a numerical value throughout the specification and the claims denotes an interval of accuracy, familiar and acceptable to a person skilled in the art. In general, such interval of accuracy is ±10%. Thus, “about ten” means 9 to 11. All numbers in this description indicating amounts, ratios of materials, physical properties of materials, and/or use are to be understood as modified by the word “about,” except as otherwise explicitly indicated.

In one embodiment of the present invention, a process for preparation of stable and highly pure triheptanoin is provided, as shown in Scheme-1 below, which includes the steps of:
a) reacting heptanoic acid with glycerol using at least one first organic solvent and at least one catalyst under reflux conditions;
b) isolating crude triheptanoin;
c) purifying crude triheptanoin using at least one adsorbing agent and optionally adding at least one second organic solvent; and
d) isolating stable and highly pure triheptanoin.

In certain embodiments of the present invention, step (c) further includes the steps of:
i) filtering followed by distilling the second organic solvent;
ii) optionally adding at least one third organic solvent and distilling the same;
iii) optionally adding a medium chain fatty acid; and
iv) optionally removing traces of the organic solvents used in the step (a) and/or step (c); and
v) isolating stable and highly pure triheptanoin with having an acid value of greater than about 0.2 mg KOH/gm.

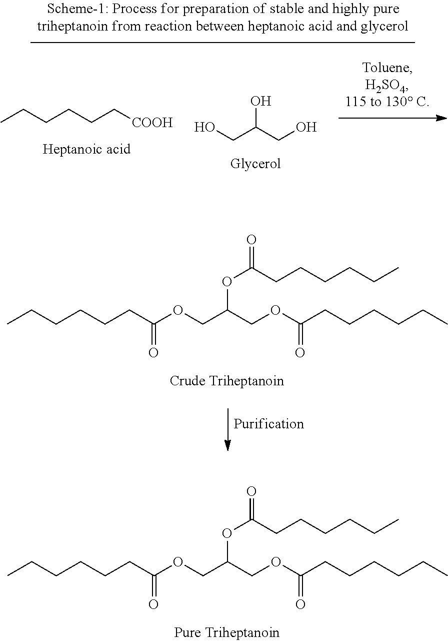

The first organic solvent used in step (a) may be selected from, but is not limited to, water immiscible organic solvents, including, but not limited to, toluene, xylene, benzene, cyclohexane, or mixtures thereof. Preferably, toluene, xylene, or mixtures thereof are used, and more preferably toluene is used in this step.

The catalyst used in step (a) may be selected from acid catalyst, basic catalyst or metal oxide catalyst. The acid catalyst may be selected from sulfuric acid, p-toluene sulfonic acid, methanesulfonic acid, phosphoric acid, camphor sulfonic acid, or mixtures thereof. In some preferred embodiments, the catalyst used is sulfuric acid, p-toluene sulfonic acid or mixtures thereof, and more preferably, sulfuric acid is used in this step.

The second organic solvent used in step (c) may be selected from water immiscible organic solvents, including, but not limited to toluene, ethyl acetate, hexane, heptane, cyclohexane, methyl tert-butyl ether, diisopropyl ether, or a mixture thereof. In some preferred embodiments, toluene, cyclohexane, heptane, hexane, or mixtures thereof are used. In more preferred embodiments, cyclohexane or heptane is used in this step.

The adsorbing agent used in step (c) may be selected from silica gel, acidic silica, neutral silica, acidic resin, neutral resin, basic resin, alumina, or mixtures thereof. Preferably, the adsorbing agent is acidic silica, neutral silica, or mixtures thereof, and more preferably, silica gel is used in this step.

The third organic solvent may be selected from dichloromethane, methyl acetate, ethylacetate, toluene, xylene, methyl tert-butyl ether, diisopropyl ether, hexane, heptane, cyclohexane, acetonitrile, water, or mixtures thereof. In some preferred embodiments, the third organic solvent is acetonitrile.

The medium chain fatty acid may be selected from heptanoic acid, hexanoic acid, stearic acid, palmitic acid, ascorbic acid, or mixtures thereof. In some preferred embodiments, the medium chain fatty acid is heptanoic acid.

The acid value of the stable triheptanoin prepared by the method of the present invention is greater than about 0.2 mg KOH/gm.

Another embodiment of the present invention provides a process for preparation of highly pure triheptanoin, which includes the steps of:
a) reacting glycerol with heptanoic acid using at least one first organic solvent and at least one acid catalyst under reflux conditions;
b) separating an organic layer from a reaction mass of step (a) and treating the reaction mass with at least one adsorbing agent and optionally adding at least one second organic solvent; and
c) isolating stable and highly pure triheptanoin.

In another embodiment of the present invention, a process for preparation of highly pure triheptanoin includes the steps of:
a) reacting heptanoic acid with glycerol using at least one first organic solvent and at least one catalyst under reflux conditions to produce an organic layer;
b) separating the organic layer from a reaction mass of step (a) and treating the reaction mass with at least one adsorbing agent and optionally adding at least one second organic solvent to produce a reaction mass;
c) filtering the reaction mass;
d) distilling the organic solvent(s) from a product of step (c); and
e) isolating stable and highly pure triheptanoin product.

In another embodiment of the present invention, a process for preparation of stable and highly pure triheptanoin includes the steps of:
a) reacting heptanoic acid with glycerol using at least one first organic solvent and at least one catalyst under reflux conditions to produce a reaction mass;
b) separating an organic layer from the reaction mass of step (a) and treating the reaction mass with at least one adsorbing agent and optionally adding at least one second organic solvent;
c) distilling the organic solvent(s);
d) stirring the obtained reaction mass;
e) filtering the reaction mass; and
f) isolating stable and highly pure triheptanoin product.

The first organic solvent used in step (a) may be selected from water immiscible organic solvents, including, but not limited to, toluene, xylene, benzene, cyclohexane, or mixtures thereof. Preferably, toluene, xylene, or mixtures thereof is used, and more preferably, toluene is used.

The catalyst used in step (a) may be selected from acid catalyst, basic catalyst or metal oxide catalyst. In some embodiments, acid catalyst may be selected from sulfuric acid, p-toluene sulfonic acid, methanesulfonic acid, phosphoric acid, camphor sulfonic acid, or mixtures thereof. In preferred embodiments, sulfuric acid, p-toluene sulfonic acid, or mixtures thereof may be used, and in more preferred embodiments, sulfuric acid is used.

The second organic solvent used in step (b) may be selected from water immiscible organic solvents, including, but not limited to, toluene, ethyl acetate, hexane, heptane, cyclohexane, methyl tert-butyl ether, diisopropyl ether, or a mixture thereof. Preferably, toluene, cyclohexane, heptane, hexane, or a mixture thereof is used, and more preferably, cyclohexane or heptane is used.

The adsorbing agent used in step (b) may be selected from silica gel, acidic silica, neutral silica, acidic resin, neutral resin, basic resin, alumina or mixtures thereof. In some preferred embodiments, silica gel, acidic silica, neutral silica, alumina or mixtures thereof is used, and in some more preferred embodiments, silica gel is used.

Surprisingly, during the stability study of the present invention, the inventors have observed that there is no impact on stability and purity of the synthesized triheptanoin, which has an acid value of greater than about 0.2 mg KOH/gm. The stable triheptanoin prepared by present invention has purity of greater than about 99.7% as measured by GC and/or HPLC.

There are two major impurities—i.e., 1,2-diester impurity and 1,3-diester impurity—formed during the synthesis as well as during the storage of triheptanoin. The structure of both impurities is as follows:

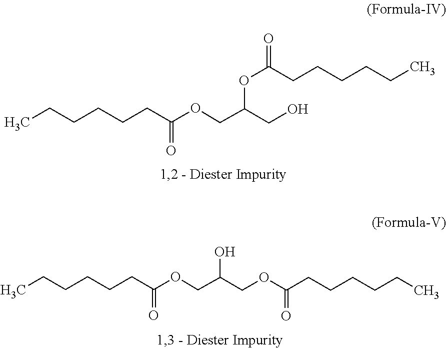

The stability data of the present invention at different stability condition(s) from initial to up to 6 (six) months is described below in Table-I:

TABLE I
Stability data of triheptanoin prepared according to examples 16 and 17

| | | | | 1,2 | 1,3 | |
|---|---|---|---|---|---|---|
| | | | | Diester | Diester | |
| | | | Acid | Impurity | Impurity | |
| | | | value | (%) | (%) | Triheptanoin |
| Sr. | Stability | Stability | (mg | (NMT | (NMT | purity |
| No. | Station | Time | KOH/gm) | 0.05%) | 0.05%) | (%) |
| Example | — | Initial | 0.21 | 0.004 | 0.002 | 99.76 |
| 16 | 25° C./60% RH | 1 M | 0.24 | 0.004 | 0.002 | 99.83 |
| | | 3 M | 0.23 | 0.007 | 0.002 | 99.82 |
| | | 6 M | 0.27 | 0.005 | 0.009 | 99.81 |
| | 40° C./75% RH | 1 M | 0.24 | 0.005 | 0.003 | 99.83 |
| | | 2 M | 0.24 | 0.008 | 0.005 | 99.78 |
| | | 3 M | 0.24 | 0.013 | 0.007 | 99.82 |
| | | 6 M | 0.26 | 0.018 | 0.015 | 99.74 |
| | 50° C. | 1 M | 0.24 | 0.008 | 0.005 | 99.83 |
| | | 2 M | 0.24 | 0.014 | 0.010 | 99.76 |
| | | 3 M | 0.25 | 0.014 | 0.012 | 99.80 |
| Example | — | Initial | 0.21 | 0.003 | 0.002 | 99.77 |
| 17 | 25° C./60% RH | 1 M | 0.24 | 0.004 | 0.002 | 99.82 |
| | | 3 M | 0.22 | 0.004 | 0.003 | 99.84 |
| | | 6 M | 0.27 | 0.003 | 0.004 | 99.82 |
| | 40° C./75% RH | 1 M | 0.24 | 0.004 | 0.004 | 99.82 |
| | | 2 M | 0.25 | 0.006 | 0.003 | 99.81 |
| | | 3 M | 0.23 | 0.006 | 0.005 | 99.82 |
| | | 6 M | 0.29 | 0.011 | 0.014 | 99.81 |
| | 50° C. | 1 M | 0.24 | 0.005 | 0.004 | 99.81 |
| | | 2 M | 0.25 | 0.008 | 0.007 | 99.78 |
| | | 3 M | 0.24 | 0.010 | 0.010 | 99.83 |

As shown in Table-I, two batches (Example 16 & Example 17) of triheptanoin prepared by the present method with acid values of greater than about 0.2 mg KOH/gm were studied for stability. The samples were kept at 25° C./60% RH, 40° C./75% RH for up to 6 months and at 50° C. for up to 3 months. During the stability study, the inventors of the present invention have focused on % of 1,2-Diester Impurity, % of 1,3-Diester Impurity and purity of triheptanoin. As seen from the data in Table-I, for triheptanoin having acid value greater than about 0.2 mg KOH/gm, the 1,2-Diester and 1,3-Diester impurities are not increasing during the storage and also assay potency of triheptanoin is maintained during the storage. The purity of triheptanoin remains constant at greater than about 99.7% as measured by GC and/or HPLC.

Furthermore, in the present invention, pure heptanoic acid is prepared from commercially available heptanoic acid via formation and purification of methyl heptanoate compound. The obtained pure heptanoic acid has purity of greater than about 99.9% as measured by high performance liquid chromatography (HPLC) and/or gas chromatography (GC).

Yet another embodiment of the present invention provides a purification of heptanoic acid (Scheme-2), which includes the steps of:
a) treating crude heptanoic acid with aqueous solution of base followed by washing with at least one first organic solvent;
b) acidifying an aqueous layer of step (a) by using a diluted acid followed by extraction in at least one second organic solvent and distilling the second organic solvent to get crude heptanoic acid;
c) esterifying the crude heptanoic acid as obtained in step (b) by using alcohol in presence of a catalyst;
d) distilling alkyl heptanoate obtained in step (c) optionally under vacuum to get pure alkyl heptanoate having a content of any impurity below about 0.1%;
e) hydrolyzing a pure heptanoate ester obtained in step (d) by using a base and at least one third organic solvent to obtain pure heptanoic acid having a purity of greater than about 99.9% and a content of any impurity below about 0.1%.

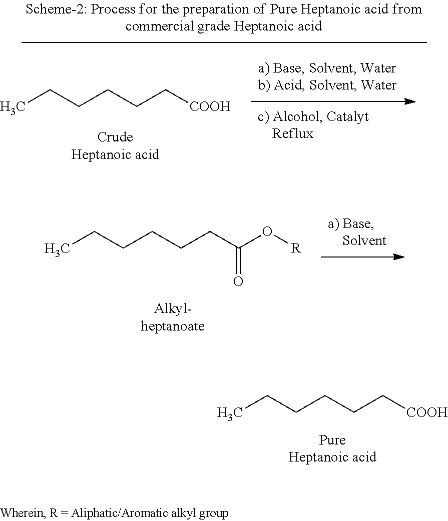

Yet another embodiment of the present inventions is to provide a process for purification of heptanoic acid comprising the steps of:
a) esterifying a heptanoic acid with an alcohol in presence of at least one catalyst;
b) distilling the crude alkyl heptanoate obtained in step (a) optionally under vacuum to get pure alkyl heptanoate having content of any impurity below about 0.1%; and
c) hydrolysing a pure alkyl heptanoate obtained in step (b) with a basic solution to obtain pure heptanoic acid having a purity of greater than about 99.9% and content of any impurity below about 0.1%.

In some embodiments of the present invention step (c) optionally includes the addition of suitable organic solvent.

The suitable solvent optionally used in step (c) may include, but is not limited to, water, dichloromethane, toluene, xylene, methyl tert-butyl ether, diisopropyl ether, hexane, heptane, cyclohexane, acetonitrile, acetone, methanol, tetrahydrofuran, acetonitrile, dioxan, or mixtures thereof may be used. In preferred embodiments, water, dichloromethane, or mixtures thereof may be used.

The alkyl heptanoate prepared during purification of heptanoic acid may include, but is not limited to, methyl heptanoate, ethyl heptanoate, propyl heptanoate, butyl heptanoate, or mixtures thereof. Preferably, alkyl heptanoate is selected from methyl heptanoate, ethyl heptanoate, or mixtures thereof. More preferably, alkyl heptanoate is methyl heptanoate.

The first, second and third solvent used for the purification of heptanoic acid may include, but is not limited to, water, dichloromethane, toluene, xylene, methyl tert-butyl ether, diisopropyl ether, hexane, heptane, cyclohexane, acetonitrile, acetone, tetrahydrofuran, dioxan, or mixtures thereof may be used. In preferred embodiments, toluene, dichloromethane, cyclohexane, or mixtures thereof may be used.

The base used for the purification of heptanoic acid may include, but is not limited to, sodium hydroxide, potassium hydroxide, potassium carbonate, sodium carbonate, cesium carbonate, lithium hydroxide, lithium carbonate, or mixtures thereof. In some preferred embodiments, sodium hydroxide may be used.

The acid used for the purification of heptanoic acid may include, but is not limited to, hydrochloric acid, hydrobromic acid, sulfuric acid, phosphoric acid, propionic acid, acetic acid, formic acid, or mixtures thereof may be used. In some preferred embodiments, sulfuric acid or hydrochloric acid may be used.

The catalyst used for the purification of heptanoic acid may include, but is not limited to, sulfuric acid, phosphoric acid, p-toluensulfonic acid, methanesulfonic acid, thionyl chloride, or mixtures thereof may be used. In some preferred embodiments, sulfuric acid may be used.

The alcohol used for the purification of heptanoic acid may include, but is not limited to, substituted or unsubstituted aliphatic alcohol.

### EXAMPLES

### Example 1: Preparation of Crude Triheptanoin

Glycerol (30 gm) and n-heptanoic acid (212 gm, 5 mol. equiv.) were added to toluene (450 ml). The reaction temperature was raised to 30 to 40° C. and stirred. Concentrated sulfuric acid (1.6 gm) was added in the reaction mass and stirred. The reaction was heated to about 110 to 140° C. (bath temperature) with a Dean Stark trap and stirred for 22 to 32 hours at this temperature. Water was collected from the azeotrope during this azeotropic reflux. The reaction mass was cooled to 20 to 30° C. and diluted with toluene (930 ml). The reaction mass was then washed with 10% sodium carbonate solution (750 ml) four times followed by sodium chloride solution wash (750 ml) two times. The organic layer was separated, and solvent was distilled from the separated organic layer to get crude triheptanoin. The resulting crude triheptanoin has the following characteristics: weight of oil: 127 gm, yield: 91%, purity (GC): 98.18%, purity (HPLC): 98.34%

### Example 2: Purification of Triheptanoin

10 gm of crude triheptanoin obtained in Example 1 was dissolved in n-heptane (100 ml) and stirred with 100-200 mesh size of silica gel (20 gm) for 1 to 2 hours at 20 to 30° C. The mass was filtered and washed with n-heptane (20 ml). Solvent was distilled out from the clear filtrate under vacuum to get pure triheptanoin with the following characteristics: weight of oil: 5.1 gm, yield: 51%, purity (GC): 99.29%, purity (HPLC): 99.12%

### Example 3: Purification of Triheptanoin

10 gm of crude triheptanoin obtained in Example 1 was dissolved in cyclohexane (100 ml) and stirred with silica gel (20 gm) for 1 to 2 hours at 20 to 30° C. The mass was filtered and washed with cyclohexane (20 ml). Solvent was distilled out from the clear filtrate under vacuum to get pure triheptanoin with the following characteristics: weight of oil: 5.2 gm, yield: 52%, purity (GC): 99.25%, purity (HPLC): 99.1%

### Example 4: Purification of Triheptanoin

10 gm of crude triheptanoin obtained in Example 1 was dissolved in cyclohexane (100 ml) and stirred with silica (20 gm) for 1 to 2 hours at 45 to 50° C. The reaction mass was filtered and washed with cyclohexane (20 ml). Solvent was distilled out from the clear filtrate under vacuum to get pure triheptanoin with the following characteristics: weight of oil: 5.1 gm, yield: 51%, purity (HPLC): 99.19%

### Example 5: Purification of Triheptanoin

10 gm of crude triheptanoin obtained in Example 1 was dissolved in toluene (100 ml) and stirred with silica (20 gm) for 1 to 2 hours at 20 to 30° C. The reaction mass was filtered and washed with toluene (20 ml). Solvent was distilled out from the clear filtrate under vacuum to get pure triheptanoin with the following characteristics: weight of oil: 6.4 gm, yield: 64%, purity (HPLC): 98.99%, purity (GC): 99.05%

### Example 6: Purification of Triheptanoin

10 gm of crude triheptanoin obtained in Example 1 was dissolved in n-heptane (100 ml) and stirred with basic alumina (20 gm) for 1 to 2 hours at 20 to 30° C. The reaction mass was filtered and washed with n-heptane (20 ml). Solvent was distilled out from the clear filtrate under vacuum to get pure triheptanoin having the following characteristics: weight of oil: 8 gm, yield: 80%, purity (GC): 99.10%, purity (HPLC): 98.51%,

### Example 7: Purification of Triheptanoin

10 gm of crude triheptanoin obtained in Example 1 was dissolved in cyclohexane (100 ml) and stirred with basic alumina (40 gm) for 1 to 2 hours at 20 to 30° C. The reaction mass was washed with cyclohexane (20 ml). Solvent was distilled out from the clear filtrate under vacuum to get pure triheptanoin having the following characteristics: weight of oil: 6.2 gm, yield: 62%, purity (HPLC): 99.10%,

### Example 8: Preparation of Methyl Heptanoate

Step (a): 300 gm heptanoic acid (purity by GC: 99.52%) was dissolved in aqueous sodium hydroxide solution (100 gm sodium hydroxide, in 1000 mL water) and washed with toluene (1800 mL). The basic aqueous layer was separated and acidified by using a dilute hydrochloric acid solution (425 mL). The heptanoic acid was extracted in toluene (3000 mL) and distilled to get heptanoic acid with the following characteristics: weight: 288 gm, yield: 96%, purity (GC): 99.70%.

Step (b): 200 gm heptanoic acid obtained in step (a) was reacted with methanol (1000 mL) in presence of sulfuric acid (7.52 gm) at 60 to 65° C. and stirred for up to about 4 hours. Methanol was distilled out from the reaction mass and cooled. Concentrated mass was dissolved in dichloromethane and washed with water, a diluted sodium bicarbonate solution followed by water wash. Solvent was distilled out from the organic layer under vacuum to get crude methyl heptanoate (weight: 178 gm). 60 gm of crude methyl heptanoate was distilled under vacuum at 35 to 70° C. and initial fraction of 5 gm was collected and then fraction of 12 gm was collected. Vacuum was removed and a residue remaining in the distillation flask was pure methyl heptanoate (37 gm).

### Example 9: Preparation of Pure Heptanoic Acid

36 gm of methyl heptanoate obtained in Example 8 was reacted with sodium hydroxide (20 gm) in water (220 mL) at 50 to 60° C. Reaction mass was then cooled to 20-30° C. and washed with dichloromethane (220 mL). The aqueous layer was separated and acidified by using dilute hydrochloric acid solution (95 mL) and heptanoic acid was extracted in dichloromethane (400 mL). The solvent was distilled out from the organic layer under vacuum to get pure heptanoic acid with the following characteristics: weight: 31.5 gm, yield: 96.8%, purity (GC): 99.94%.

### Example 10: Preparation of Triheptanoin

4 gm of glycerol and pure heptanoic acid (28.3 gm, 5 mol. equiv.) obtained in Example 9 were charged in toluene (60 ml). The temperature was raised to 30 to 40° C. and the reaction mass was stirred. Concentrated sulfuric acid (0.21 gm) was added in the reaction mass and stirred. The reaction was heated to 110 to 140° C. (bath temperature) with a Dean Stark trap and stirred for 22 to 32 hours at this temperature. Water was collected from the azeotrope during azeotropic reflux. Reaction mass was cooled to 20 to 30° C. and diluted with toluene (140 ml). Reaction mass was then washed with 10% sodium carbonate solution (100 ml) followed by sodium chloride solution wash (100 ml). The organic layer was separated, and solvent was distilled from the separated organic layer to get crude triheptanoin with the following characteristics: weight of oil: 16.9 gm, yield: 90.8%, purity (GC): 98.51%.

### Example 11: Purification of Triheptanoin

7 gm crude triheptanoin obtained in Example 10 was dissolved in cyclohexane (70 ml) and stirred with silica (14 gm) for 1 to 2 hours at 20 to 30° C. The reaction mass was filtered and washed with cyclohexane (14 ml). Solvent was distilled out from the clear filtrate under vacuum to get the pure triheptanoin with the following characteristics: weight of oil: 3.6 gm, yield: 51.5%, purity (GC): 99.70%.

### Example 12: Preparation of Methyl Heptanoate

Heptanoic Acid (2 kg) was charged in Methanol (10 Lit) and cooled. Sulfuric acid (0.3 Kg) was added and then mass stirred at 55 to 65° C. Then solvent was distilled out under vacuum. Concentrated mass dissolved in dichloromethane (10 Lit) and washed with purified water (8 Lit) followed by wash of Sodium bicarbonate solution (8 Lit) followed by purified water (8 Lit). Solvent distilled out under atmospheric pressure from the combined organic layer. After that the temperature was raised up to 95° C., and product was distilled out under vacuum and collected into fractions. Wt. of Methyl Heptanoate (1480 gm, 67%). GC purity: 99.8%

### Example 13: Preparation of Pure Heptanoic Acid

Methyl Heptanoate (1300 gm) obtained in Example 12 was reacted with sodium hydroxide (55.5 gm) in water at 50 to 60° C. Reaction mass was cooled to room temperature and stirred with dichloromethane (2600 ml). The aqueous layer was acidified using hydrochloric acid solution, and product was extracted in dichloromethane (6500 ml). Initially, solvent was distilled out atmospherically and then under vacuum at 50 to 60° C. to get pure Heptanoic Acid. Yield: 1092 gm, 93%. GC purity: 99.95%.

### Example 14: Preparation of Crude Triheptanoin

Glycerol (150 gm) was reacted with heptanoic acid (1060 gm) obtained in Example 13 in presence of sulfuric acid (12.8 gm) in toluene (2250 ml) under Azeotropic reflux at 110 to 130° C. with a Dean-Stark trap. Then, the reaction mass was cooled to room temperature and washed with water followed by aqueous sodium carbonate wash and aqueous sodium chloride wash. The organic layer was then treated with activated carbon and silica gel 100-200 mesh. Filtrate was collected and washed with aqueous sodium bicarbonate solution and then aqueous sodium chloride solution. Solvent was distilled out from the organic layer under vacuum at 50 to 65° C. to get crude Triheptanoin. Yield: 495 gm, GC Purity: 99.5%.

### Example 15: Preparation of Pure Triheptanoin

Crude Triheptanoin (350 gm) obtained in Example 14 was dissolved in cyclohexane (3500 ml) and was stirred with 100-200 mesh silica gel. After filtration, filtrate was again stirred with 100-200 mesh silica gel and the same operation was repeated one more time. The filtrate was then washed with sodium bicarbonate, water and sodium chloride solution. The organic layer was treated with activated carbon. The solvent was distilled out under vacuum from the filtrate. The reaction mass was cooled at 25 to 30° C. and charged acetonitrile in the reaction mass followed by cooling at 0 to 5° C. Then, the reaction mass stirred for about 30 to 50 minutes, and the solvent was filtered and distilled out under vacuum to get concentrated reaction mass. Heptanoic acid (0.0915 gm) was charged into the mass and then nitrogen gas was purged through the mass at 50 to 60° C. Finally, the mass was cooled and collected to get pure Triheptanoin. Yield: 180.9 gm, 51.7%. HPLC purity: 99.8%

### Examples 16 and 17: Preparation of Pure Triheptanoin

By following the same procedures as Examples 12 to 15, two additional batches of Triheptanoin were prepared (Examples 16 and 17). The stability results of triheptanoin obtained in Examples 16 and 17 are disclosed in Table-I.

It should be noted that the invention in its broader aspects is not limited to the specific details, representative compositions, methods, and processes, and illustrative examples described in connection with the preferred embodiments and preferred methods. Modifications and equivalents will be apparent to practitioners skilled in this art and are encompassed within the spirit and scope of the appended claims.

What is claimed is:

1. A process for preparation of a pharmaceutically acceptable triheptanoin, comprising the steps of:
a) reacting glycerol with heptanoic acid using at least one first organic solvent and at least one catalyst under reflux conditions;
b) isolating crude triheptanoin;
c) purifying the crude triheptanoin using at least one adsorbing agent and adding at least one second organic solvent, and adding a medium chain fatty acid; and
d) isolating the pharmaceutically acceptable triheptanoin;
wherein the pharmaceutically acceptable triheptanoin has a purity of about 99% or more and an acid value of about 0.2 mg KOH/gm or more.

2. The process according to claim 1, wherein the process does not involve column chromatography.

3. The process according to claim 1, wherein the at least one first organic solvent and the at least one second organic solvent are selected from one or more water immiscible organic solvents.

4. The process according to claim 3, wherein the one or more water immiscible organic solvents is selected from toluene, xylene, benzene, ethyl acetate, hexane, heptane, cyclohexane, methyl tert-butyl ether, diisopropyl ether, 2-methyl tetrahydrofuran and mixtures thereof.

5. The process according to claim 4, wherein the at least one first organic solvent is toluene.

6. The process according to claim 1, wherein the at least one catalyst is selected from an acid catalyst and a basic catalyst.

7. The process according to claim 6, wherein the acid catalyst is selected from sulfuric acid, p-toluene sulfonic acid, methanesulfonic acid, phosphoric acid, camphor sulfonic acid, thionyl chloride, and mixtures thereof.

8. The process according to claim 7, wherein the acid catalyst is sulfuric acid.

9. The process according to claim 1, wherein the at least one adsorbing agent is selected from silica, acidic silica, neutral silica, acidic resin, neutral resin, basic resin and mixtures thereof.

10. The process according to claim 9, wherein the at least one adsorbing agent is silica.

11. The process according to claim 1, wherein the medium chain fatty acid is selected from heptanoic acid, hexanoic acid, stearic acid, palmitic acid, ascorbic acid, and mixtures thereof.

12. The process according to claim 11, wherein the medium chain fatty acid is heptanoic acid.

13. The process according to claim 1, wherein step (a) is performed at a temperature of about 140° C. or less.

14. A process for preparation of a pharmaceutically acceptable triheptanoin, comprising the steps of:
a) reacting glycerol with heptanoic acid using at least one first organic solvent and at least one catalyst under reflux conditions at a temperature of about 140° C. or less to obtain crude triheptanoin;
b) purifying the crude triheptanoin using at least one adsorbing agent and adding at least one second organic solvent; and
c) isolating the pharmaceutically acceptable triheptanoin having a purity of at least about 99.5%.

15. The process according to claim 1, wherein the heptanoic acid used in step a) is prepared by:
esterifying crude heptanoic acid with an alcohol in presence of the at least one catalyst to obtain crude alkyl heptanoate;
distilling the crude alkyl heptanoate to obtain pure alkyl heptanoate; and
hydrolyzing the pure alkyl heptanoate with a base and at least one third organic solvent to obtain the heptanoic acid;
wherein the heptanoic acid has a purity of greater than about 99.5% and a content of any impurity of about 0.1% or below.

16. The process according to claim 15, wherein the base is selected from sodium hydroxide, potassium hydroxide, potassium carbonate, sodium carbonate, cesium carbonate, lithium hydroxide, lithium carbonate, and mixtures thereof.

17. The process according to claim 15, wherein the acid is selected from hydrochloric acid, hydrobromic acid, sulfuric acid, phosphoric acid, propionic acid, acetic acid, formic acid, and mixtures thereof.

18. The process according to claim 15, wherein the alcohol is selected from substituted or unsubstituted aliphatic alcohol and aromatic alcohol.

19. The process according to claim 15, wherein the at least one third organic solvent is selected from water, dichloromethane, toluene, xylene, methyl tert-butyl ether, diisopropyl ether, hexane, heptane, cyclohexane, acetonitrile, acetone, methanol, tetrahydrofuran, acetonitrile, dioxan, and mixtures thereof.

20. The process according to claim 15, wherein alkyl heptanoate is selected from methyl heptanoate, ethyl heptanoate, propyl heptanoate, butyl heptanoate, and mixtures thereof.
